SEQUENCE LISTING (2 sequences)

SEQ ID NO: 1 - protein, 15 aa, synthetic construct
FGLATEKSRWSGSHQ

SEQ ID NO: 2 - protein, 15 aa, synthetic construct
LATEKSRWSGSHQFE

(19) United States
(12) Patent Application Publication
(10) Pub. No.: US 2025/0299838 A1
(43) Pub. Date: Sep. 25, 2025

(54) OPTIMIZING VACCINE PRODUCTION THROUGH SIMULATION

(71) Applicant: AMAZON TECHNOLOGIES, INC., Reno, NV (US)

(72) Inventors: Samuel Anthony DANZIGER, Seattle, WA (US); Haibao TANG, Cupertino, CA (US); Alena HARLEY, Los Gatos, CA (US); Layne Christopher PRICE, Seattle, WA (US); Frank Wilhelm SCHMITZ, Seattle, WA (US); Antje HEIT, Seattle, WA (US); David HECKERMAN, Bellevue, WA (US); Anta IMATA SAFO, Seattle, WA (US); Brandon Yacullo HOANE, Seattle, WA (US); Sean Michael STOCKWELL, Seattle, WA (US); Beshoy SARKIS, Bothell, WA (US)

(21) Appl. No.: 18/612,632

(22) Filed: Mar. 21, 2024

(51) Int. Cl. G16H 70/40; G16B 15/30; G16B 40/20

(52) U.S. Cl. CPC G16H 70/40; G16B 15/30; G16B 40/20

(57) ABSTRACT

Methods and systems are disclosed for selecting a set of peptides from a plurality of peptides for producing a drug product. A request may be received to produce a vaccine that meets specific requirements, including the desired immune response and the inclusion of certain types of peptides. The system may rank peptides using one or more metrics that factor in immunogenicity and/or manufacturability. Based on the ranking, the system may select a group of peptides for inclusion in a manufacturing simulation process, which returns a set of peptides that are predicted to be successfully manufactured. The system refines its selection to a subset of manufacturable peptides based on specific criteria. This iterative process continues until predefined conditions are met, such as the convergence of the simulation results. Based on these results, the system identifies an optimal or near-optimal set of peptides that can be used for effective drug production.

### SEQUENCE LISTING

The instant application contains a Sequence Listing which has been submitted electronically in XML format and is hereby incorporated by reference in its entirety. Said XML file, created on Mar. 13, 2024, is named 146401.093459_SL.xml and is 2,572 bytes in size.

### BACKGROUND

Vaccines play a crucial role in public health. They are designed to provide protection against diseases by exposing the immune system to antigens (usually proteins) produced by a pathogen or tumor with the goal of training an effective adaptive immune response. One way to produce these life-saving vaccines involves creating protein fragments called peptides. Some vaccines directly include synthetic peptides, while others include synthetic nucleic acids that will produce peptides after injection. These peptides, which are sequences of amino acids, act as antigens to trigger an immune response without causing the disease. One of the challenges in vaccine production is selecting the right combination of peptides that not only are capable of activating an immune response, but also can be manufactured with high success rates. Therefore, determining the optimal combination of peptides that are both manufacturable and effective presents a significant challenge in vaccine development. Consequently, there is a need for a more efficient method to determine optimal combinations of peptides or nucleic acids for use in vaccine manufacturing.

### SUMMARY OF THE INVENTION

Methods and systems are disclosed for intelligently selecting a set of peptides from a multitude of peptides for producing a drug product. The methods can include the steps of receiving a request for selecting peptides (e.g., one or more peptides) from a plurality of peptides to produce a drug product; receiving data including information on manufacturability associated with each peptide of the plurality of peptides; receiving data including information on immunogenicity associated with each peptide of the plurality of peptides; and generating, using a statistical model pipeline, a set of peptides from the plurality of peptides for producing the drug product based on the information on manufacturability and the information on a second feature; the statistical model optimizes at least one of an immunogenicity sum or a manufacturability sum associated with the set of peptides while meeting one or more criteria. The method can include the steps of ranking the plurality of peptides based on the information on manufacturability and the information on the second feature associated with each peptide, with the second feature being immunogenicity associated with each peptide; and selecting, based on the ranking and the one or more criteria, a first set of peptides from the plurality of peptides for inclusion in a simulated manufacturing process, the simulated manufacturing process being a part of the statistical model pipeline; the one or more criteria include one or more of an expected number of long peptides, an expected number of short peptides, and one or more specific target mutations that the drug is intended to be effective against. The method can include the steps of generating, based on the simulated manufacturing process, a second set of peptides; said second set of peptides is predicted to pass the simulated manufacturing process; selecting a subset of peptides from the second set of peptides based on an immunogenicity score associated with each peptide and based on the one or more criteria; and calculating an aggregated immunogenicity score for the subset of peptides. The method can include a step of repeatedly executing the simulated manufacturing process on the first set of peptides for a number of iterations, producing a number of simulation outcomes; and determining an average aggregated immunogenicity score associated with the first set of peptides based on the number of simulation outcomes. The steps can include computing a product of immunogenicity score and manufacturability score for each peptide; the information on immunogenicity includes an immunogenicity score for each peptide and the information on manufacturability includes a manufacturability score for each peptide; and the ranking is based on the product of the immunogenicity score and manufacturability score. The steps can include receiving a request for selecting one or more peptides from a plurality of peptides to produce a drug product; receiving data associated with the plurality of peptides, the received data comprising information on manufacturability and information on one or more features associated with each peptide of the plurality of peptides; and determining, using a statistical model, a set of peptides from the plurality of peptides for producing the drug product based on the information on manufacturability and the information on at least one of the one or more features.

The information on one or more features can include one or more of: information on immunogenicity, information on expected numbers of different types of peptides, and information on specific target mutations that the drug is intended to be effective against associated with each peptide. Determining the set of peptides further comprises ranking the plurality of peptides based on the information on manufacturability and the information on the feature associated with each peptide; and selecting, based on the ranking and one or more criteria, a first set of peptides from the plurality of peptides for inclusion in a simulated manufacturing process. The criteria can include one or more of an expected number of long peptides, an expected number of short peptides, and one or more specific target mutations that the drug is intended to be effective against. The method can include steps of generating, based on the simulated manufacturing process, a second set of peptides; said second set of peptides can be predicted to pass the simulated manufacturing process; selecting a subset of peptides from the second set of peptides based on a feature score associated with each peptide and based on the one or more criteria; and calculating an aggregated feature score for the subset of peptides. The method can include steps of repeatedly executing the simulated manufacturing process on the first set of peptides for a number of iterations, producing the number of simulation outcomes; and determining an average aggregated feature score associated with the first set of peptides based on the number of simulation outcomes. Ranking the plurality of peptides can further include computing a product of feature score and manufacturability score for each peptide, wherein the information on at least one of the features includes a feature score for each peptide and the information on manufacturability includes a manufacturability score for each peptide; and the ranking is based on the product of the feature score and manufacturability score. Selecting the set of peptides can be based on a greedy search algorithm that optimizes a goal associated with the set of peptides, while meeting one or more criteria.

### BRIEF DESCRIPTION OF THE DRAWINGS

Various embodiments in accordance with the present disclosure will be described with reference to the drawings, in which:

FIG. 1 illustrates an exemplary system environment that includes a vaccine manufacturing simulator, in accordance with the various embodiments.

FIG. 2 illustrates an exemplary process for selecting peptides for vaccine production, in accordance with the various embodiments.

FIG. 3 illustrates an exemplary process for simulating expected immunogenicity for a group of peptides, in accordance with the various embodiments.

FIG. 4 illustrates an exemplary process for simulating peptide manufacturing, in accordance with the various embodiments.

FIG. 5 is an exemplary graph illustrating a distribution for immunogenicity sum, in accordance with the various embodiments.

FIG. 6 illustrates an exemplary process for selecting peptides by comparing multiple ranking algorithms, in accordance with the various embodiments.

FIG. 7 is an exemplary graph illustrating different distributions of immunogenicity based on different ranking algorithms, in accordance with the various embodiments.

FIG. 8 illustrates an exemplary process for selecting peptides for vaccine production, in accordance with the various embodiments.

FIG. 9 illustrates components of a computing device that can be utilized in accordance with various embodiments.

FIG. 10 illustrates an environment for implementing aspects in accordance with various embodiments.

FIG. 11 illustrates components of an environment in which aspects of various embodiments can be implemented.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Approaches described and suggested herein relate to selecting peptides for vaccine production. Methods and systems are disclosed for intelligently selecting a set of peptides from a multitude of peptides (e.g., selecting a set of 20 peptides from 100,000 peptides) for producing a drug product. A request can be received to produce a vaccine that meets specific requirements while maximizing immunogenicity, the specific requirements including the desired immune response and the inclusion of certain types of peptides (e.g., short peptides, long peptides, a combination of short and long peptides, such as a combination of 14 long peptides and 6 short peptides). In one embodiment, the distinction between short and long peptides is defined by their amino acid count, with short peptides containing fewer amino acids than long peptides. The difference in length enables short peptides and long peptides to trigger different immune responses. For example, CD8 T-cells are usually activated by shorter peptides, typically 8-10 amino acids in length. On the other hand, CD4 T-cells are typically activated by longer peptides, such as ranging from 13-25 amino acids. The system can rank peptides using one or more metrics that factor in immunogenicity (i.e., the ability to stimulate an immune response) and/or manufacturability (i.e., the likelihood of successful production). Based on this ranking of the peptides, the system can select a group of peptides to go through a manufacturing simulation process, which will return a set of peptides that are predicted to be successfully manufactured. Following the simulation, the system can refine its selection to a subset of these manufacturable peptides based on specific criteria, such as peptide types or peptides that cover certain mutations, while focusing on maximizing immunogenicity. This iterative process can continue until one or more predefined conditions are met, such as the convergence of the simulation results. Based on these results, the system can identify an optimal or near-optimal set of peptides that can be used for effective drug production. In one embodiment, the disclosed methods and systems may apply to not only peptides but also other biomolecules such as deoxyribonucleic acid (DNA), and ribonucleic acid (RNA) that exhibit non-random and predictable manufacturing failure rates. Methodologies described herein for efficient evaluation and optimizing production processes may also be applicable to such biomolecules.

The disclosed systems and methods for simulating a drug production process provide multiple technical advantages. For example, the simulating system can enhance efficiency and accuracy in selecting peptides for vaccine production. By using simulation algorithms, the system can efficiently filter a multitude of peptides and evaluate the peptides based on their manufacturability and immunogenicity. The system can streamline the peptide selection process, and also ensure that the chosen peptides have a higher likelihood of successful production and effectiveness in stimulating an immune reaction. This targeted approach can reduce the time and resources spent on currently available trial-and-error methods, leading to faster development cycles for vaccines.

Additionally, the system can significantly lower the likelihood of errors. Manual computations, particularly when dealing with an extensive number of peptides, are prone to error. Traditionally, such complicated tasks have been dependent on the expertise and judgment of skilled professionals. Despite their expertise, humans can make errors, especially when faced with repetitive and complex calculations. By automating the peptide selection process, the system greatly reduces the chance of human error and produces a more reliable and accurate outcome. In the field of vaccine development, where the utmost precision is required, the consequences of even small errors can be substantial, potentially affecting thousands of lives. Automating this process can improve reliability, and also enhance the overall efficiency of vaccine development, thus providing vaccines that meet safety and efficacy standards, while minimizing potential risks of errors.

Further, the system may run and compare multiple algorithms (such as various ranking algorithms) to identify which algorithm yields the best results. The ability to fine-tune the selection process is invaluable, because even a slight improvement in selection algorithms can have profound implications for patient outcomes. By identifying the most effective algorithm, the system can provide a set of chosen peptides that have the greatest potential efficacy. Such marginal gains, when amplified across the scale of global healthcare, can lead to significant advancements in public health.

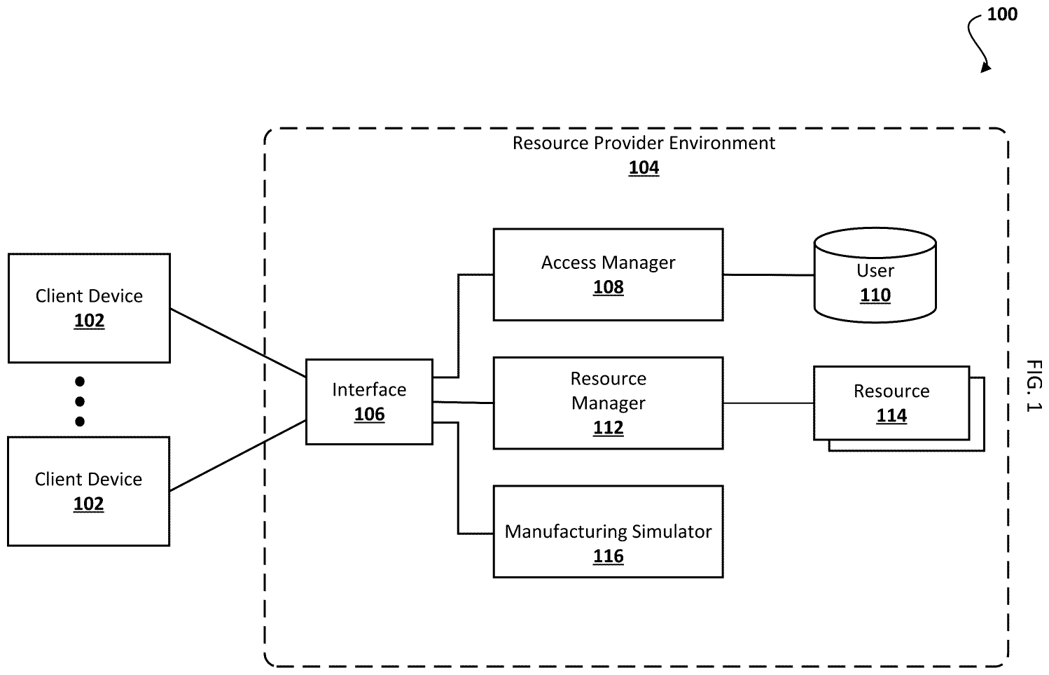

FIG. 1 illustrates an exemplary system 100 that can be used to provide functionalities related to a manufacturing simulation process to users, applications, clients, or other such entities according to at least one embodiment described herein. The exemplary system 100 can include one or more client devices 102 and a resource provider environment 104 that includes an interface 106, an access manager 108, a resource manager 112, and a manufacturing simulator 116.

In the embodiment illustrated in FIG. 1, the resource provider environment 104 can include a number of resources 114 that can be made available for use by various users. These resources can include any appropriate computing or electronic resources useful in a networked computing environment, as they relate to physical or virtual servers or compute instances, data repositories, and the like. Users can use various client devices 102 to engage in communications with these resources, such as by sending requests to be received by an interface 106 of the resource provider environment 104, where the interface 106 can direct information for the request within the environment 104 as appropriate. In at least one embodiment, information for a request will be passed to an access manager 108 that can compare information associated with a request against information stored in a user data repository 110, or other such location, to attempt to authenticate a source of the request and determine that the source is authorized to access various resources. Once authentication and authorization are verified, information for the request can be directed to a resource manager 112 to attempt to determine and allocate one or more appropriate resources 114 for serving the request. Once allocated, a request can be directed to an allocated resource instead of first being directed to a resource manager 112.

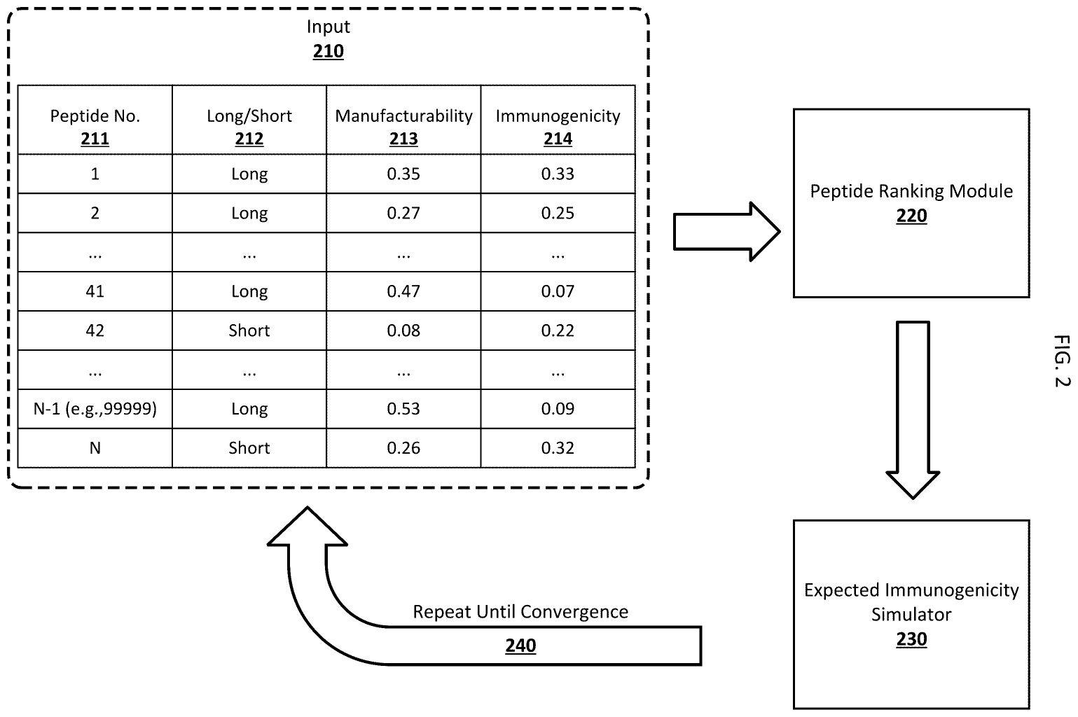

In some embodiments, a user may wish to perform a task that involves selecting a set of peptides from a population of peptides for producing a drug product. For example, the manufacturing simulator 116 can receive a request through the interface 106 from the client device 102 to select a combination of peptides for producing a drug product based on specified criteria. Information associated with the request can be directed to the manufacturing simulator 116 for performing simulation tasks. For example, the client device 102 may send a request to choose a specific number of peptides that target particular mutations, with a defined preference for long and short peptides. Upon receiving this request, the manufacturing simulator 116 can carry out simulations and selection processes tailored to these specified requirements. Functionalities associated with the manufacturing simulator 116 are discussed in greater detail in accordance with FIGS. 2-8.

FIG. 2 illustrates an exemplary process for selecting a first collection of peptides from a multitude of candidate peptides. As illustrated in FIG. 2, the initial step involves input 210, which comprises data associated with a pool of N peptides. This input 210 can comprise data associated with each peptide, such as unique identification number 211, categorization as either long or short 212, a manufacturability score 213 that indicates the likelihood a peptide is predicted to be successfully manufactured, an immunogenicity score 214, or combination thereof that indicates the likelihood that a peptide can provoke an immune response. In one embodiment, each peptide may also be associated with a solubility score that quantifies the solubility of the peptide. In one embodiment, each peptide is associated with one or more variants or mutations that are covered by the peptide window. When a variant, such as a mutation, is identified, multiple peptide sequences, also referred to as peptide windows, can cover this mutation. For example, both sequences FGLATEKSRWSGSHQ (SEQ ID NO:1) and LATEKSRWSGSHQFE (SEQ ID NO:2) cover the mutation chr7:140453136:A:T. In one embodiment, the input 210 may further include one or more mutations that the drug product is intended to cover. For example, in a particular embodiment having a tumor with 10 mutations, the input 210 can include details such as information related to these 10 mutations and which mutations are targeted by each peptide. This information concerning the mutations corresponding to each peptide window can also be included in input 210. In one embodiment, the manufacturability score 213 and immunogenicity score 214 can be determined using neural networks or other machine learning algorithms/models. In one embodiment, manufacturability scored may factor in features such as solubility of peptides such as when multiple peptides are combined. Although each peptide may be soluble individually, their combination can alter solubility characteristics (e.g., can potentially lead to insolubility). These machine learning models can take such peptide features as inputs and trained to predict these scores for each peptide.

The input 210 is passed to the peptide ranking module 220, which performs one or more ranking algorithms on the pool of N peptides and selects a first collection of P peptides (e.g., selecting 48 peptides from 100,000 candidate peptides). The initial selection can be referred to as the collection of P peptides. In one embodiment, the peptide ranking module 220 is designed to evaluate and rank peptides using a variety of metrics. This evaluation can be conducted in various ways. In one embodiment, simple metrics can be used to evaluate and rank the pool of peptides. For example, one approach is to assess the peptides solely on the basis of their immunogenicity score 214. Alternatively, the evaluation can be based on a combined metric that multiplies the immunogenicity score with the manufacturability score of each peptide (i.e., immunogenicity×manufacturability). In this embodiment, the peptide ranking module 220 can rank the peptides based on immunogenicity 214, immunogenicity 214×manufacturability 213, or even immunogenicity×solubility. After deciding on the metric to be used, the peptide ranking module 220 ranks the N peptides in the input 210. Following this ranking, a predetermined number of the highest-ranking peptides (i.e., referred to as P peptides) are selected. In one embodiment, the peptide ranking module 220 can incorporate additional constraints when selecting the initial set of P peptides. These constraints can include specific types of peptides (e.g., requiring a minimum number of long and/or short peptides) and desired mutations to be covered by the final selection of peptides. As an example, if a drug product aims to cover a tumor with 10 mutations, a goal may be to ensure the successful manufacture of peptides that can cover each of the 10 mutations. That is, a manufacturing simulator can maximize the immunogenicity of the manufactured peptides, while ensuring that all 10 mutations are covered by at least one peptide, or that all mutations meet a certain minimum immunogenicity threshold for the manufactured peptides covering them. For example, the peptide ranking module 220 can, based on a ranking metric, identify two peptides with the highest ranking. However, if both peptides target the same mutation, potentially leaving other mutations uncovered, the peptide ranking module 220, in subsequent selections, can consider not only the ranking, but also with aim to cover all mutations with at least one peptide or to achieve a threshold of immunogenicity score for each mutation.

In another embodiment, the peptide ranking module 220 can utilize an optimization algorithm such as a greedy search on the N peptides to search for an optimal or near-optimal collection P peptides subject to constraints on one or more objectives. The greedy search may focus on optimizing certain metrics involving immunogenicity score and/or manufacturability score. For example, the greedy search may maximize 1−Π(1−immunogenicityᵢ), for each peptide i in P peptides. This metric represents maximizing the probability of preserving at least one immunogenic peptide in P peptides. The greedy search can alternatively maximize Σ immunogenicityᵢ, for each peptide i in P peptides. This metric represents maximizing total immunogenicity of all peptides in the collection of P peptides.

To further elaborate the greedy search with an illustrative example, the greedy search can begin the process by initializing a set of 48 peptides (i.e., P peptides). This initial set can be selected based on a metric such as the highest individual immunogenicity scores or the highest (immunogenicity×manufacturability) scores, depending on the specific criteria chosen. In one embodiment, the initialization can be organized according to the targeted mutations that need to be covered. For example, for each mutation, one or more peptides are selected and the peptides selected for each mutation are those that yield the highest product of immunogenicity and manufacturability scores. Once the initial set of 48 peptides is established, the greedy search iteratively evaluates potential substitutions to enhance the selected objective. In each iteration, the algorithm explores the possibility of replacing one of the peptides in the current set with a different peptide from the remaining pool of candidates. The selection for substitution is based on the incremental improvement it offers towards the objectives, such as a) increasing the probability of having at least one highly immunogenic peptide, calculated 1−Π(1−immunogenicityᵢ), or b) augmenting the sum of the immunogenicity scores of the peptides in the set. The substitution is made if it improves the set with respect to the objective. In one embodiment, a non-greedy substitution algorithm can be used. Such a substitution algorithm may not select the peptide substitution that results in the most significant improvement. The algorithm can explore new options and can select a peptide substitution that results in a lower immunogenicity. The exploration can allow for possibilities of future substitutions that can collectively lead to a higher overall immunogenicity. Using such a non-greedy substitution algorithm can provide a larger benefit to patients because of the potential improvements in immunogenicity, especially when dealing with a large number of patients. This process continues, with the algorithm making one substitution at a time, until no further substitutions can be found that improve the set according to these metrics. In some embodiments, a maximum number of iterations or limited computational resources, can also serve as ending criteria. The result is a refined set of 48 peptides that best meet the specified objectives of maximizing immunogenicity or the likelihood of having highly immunogenic peptides.

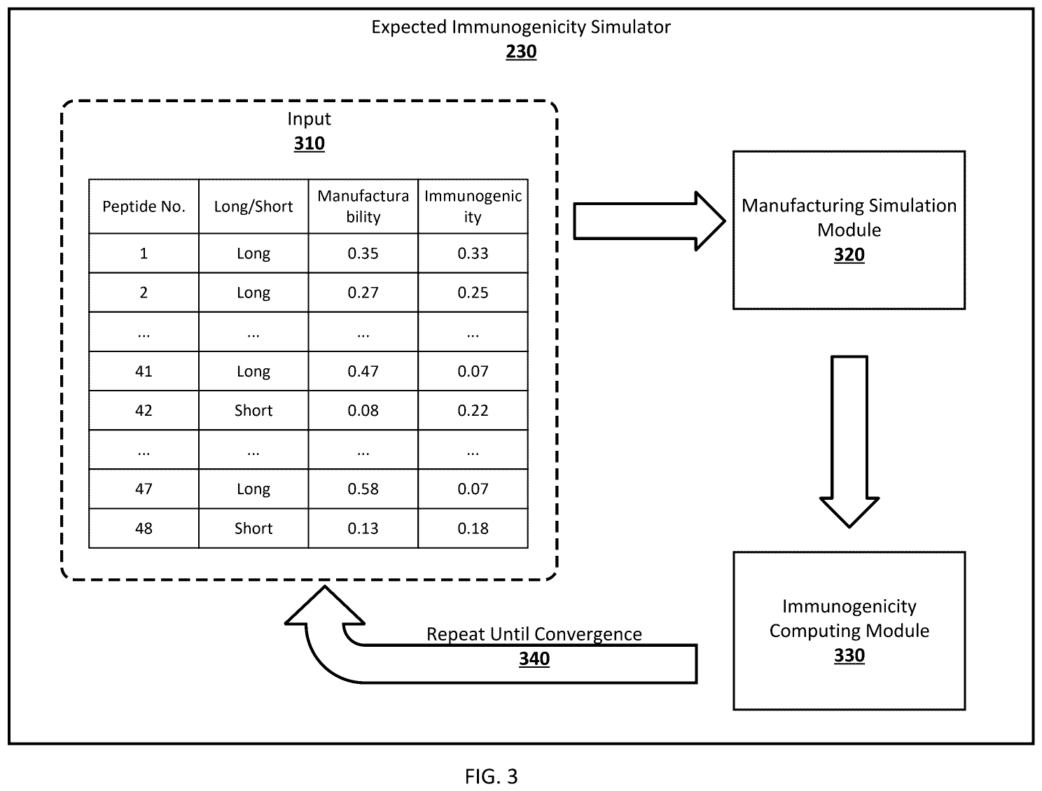

After the peptide ranking module 220 determines the initial selection of P peptides, the selected peptides can be passed to an expected immunogenicity simulator 230 that selects a subset from the P peptides. The immunogenicity simulator 230 refines the P peptides by choosing which of the manufactured peptides should be in the drug product (e.g., selecting up to 20 peptides from up to 48 peptides, which were successfully manufactured), which can be referred to as a subset S for discussion purposes. The expected immunogenicity simulator 230 can simulate a manufacturing process and select the S peptides from the peptides that pass a manufacturing process while considering other constraints, such as the mix of short/long peptides and making sure that each mutation is covered by at least one peptide. For example, the expected immunogenicity simulator 230, when determining which peptides to include in the subset S from all peptides that survived a manufacturing process, may also consider constraints such as a required number of long/short peptides. Additionally, the determination is further based on if the selection effectively covers all mutations, with each mutation covered by at least a number of peptides (e.g., each mutation is covered by at least one peptide). In one embodiment, if it is not possible to cover all mutations, a group of short/long peptides can be selected to maximize the number of mutations that can be covered. The expected immunogenicity simulator 230 can further calculate an expected immunogenicity score for the refined subset of S peptides. The expected immunogenicity simulator 230 is discussed in greater detail in accordance with FIGS. 3-5.

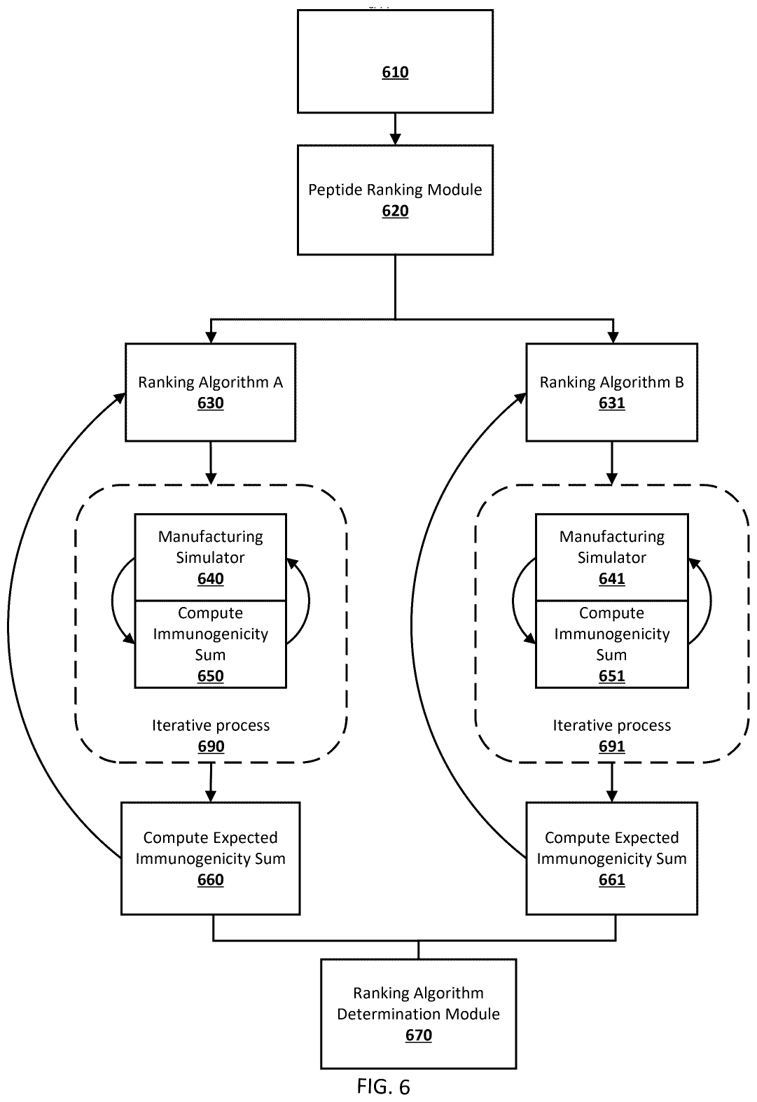

The process illustrated in FIG. 2, starting from input 210 with N peptides, to a final refinement of S peptides and a computed expected immunogenicity score for the S peptides, is also an iterative process. That is, throughout this process, both ranking and simulation algorithms are repeatedly 240 applied, and an expected immunogenicity score for the optimized group of peptides is generated for each cycle of the iteration. Such an iterative cycle continues until certain criteria are met, such as when the mean or median of the expected immunogenicity scores reaches a state of stability. Once the expected immunogenicity scores stabilize, the process concludes, and the final stable expected immunogenicity score is then considered as the score for the given ranking algorithm. Such an expected immunogenicity score represents the optimized immunogenicity of the peptides after undergoing the iterative simulation process. This iterative process for selecting and refining a set of peptides with the goal of achieving optimal immunogenicity is further discussed in accordance with FIG. 6. The detailed discussion of an immunogenicity simulator 230, including its optimization algorithm and the process for selecting a refined subset of S peptides, is discussed in greater detail in accordance with FIG. 3.

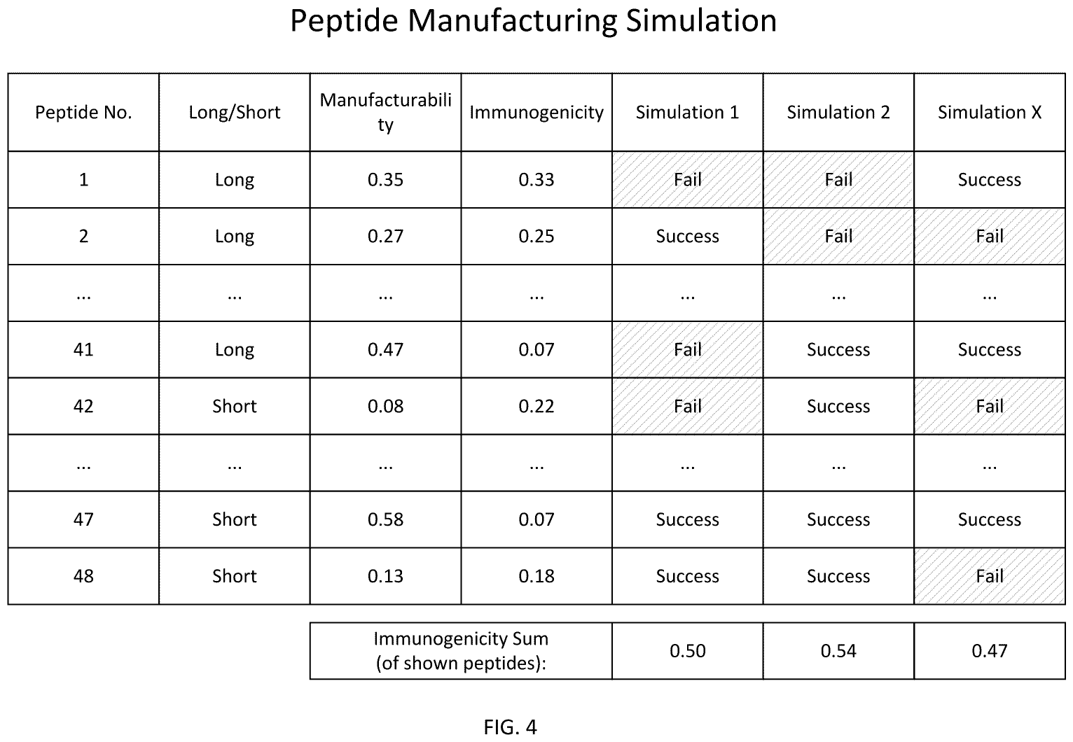

FIG. 3 illustrates an exemplary simulating process performed by the expected immunogenicity simulator 230. The expected immunogenicity simulator 230 can select a subset of refined peptides given the results from ranking algorithms (e.g., selecting S peptides from P peptides). The input 310, in this example illustrated in FIG. 3, can be a group of 48 peptides selected from N candidate peptides. Each peptide in this set is associated with specific data like peptide type, manufacturability, immunogenicity and/or solubility. The first stage of the process involves input 310 undergoing a manufacturing simulation via manufacturing simulation module 320. The module is designed to simulate the manufacturing process and predict which peptides can be successfully produced. An exemplary manufacturing simulation process performed by the manufacturing simulation module 320 is illustrated in FIG. 4. The peptides that successfully pass the manufacturing simulation are then forwarded to additional processing modules for further evaluation. An example immunogenicity computing module 330 might select as S 13 long and 7 short peptides from the peptides that are successfully manufactured such that 5 mutations are covered by at least one peptide and the overall immunogenicity of the 20 peptides is maximized.

FIG. 4 illustrates an exemplary manufacturing simulation process. The starting point of this process involves 48 peptides, each assigned a respective manufacturability score, that represents the likelihood of a peptide being successfully produced by a manufacturing process. In one embodiment, a Bernoulli trial is used for such a simulation process. A Bernoulli trial is a random experiment with two possible outcomes such as “success” and “failure.” In this context, the trial is used to determine whether each peptide passes or fails the manufacturing simulation. The process works by generating a random number based on the Bernoulli distribution (e.g., generating a random number between 0 and 1) for each peptide in every iteration of the simulation. If the number generated is greater than the manufacturability score for a peptide, the peptide is considered to have failed the simulation. Conversely, if the generated number is less than the manufacturability score, the peptide is considered to have passed the simulation and is predicted to be manufactured for the iteration.

To illustrate this process, take the example of peptide No. 1 in FIG. 4, which is associated with a manufacturability score of 0.35. In the first simulation, if, for example, a number of 0.7 is generated, the peptide is considered to have failed to be manufactured due to the generated number being higher than the manufacturability score. In a second simulation, a number of 0.5 can be generated, which results in another failure. However, in the Xth iteration, a number of 0.2 may be generated, which is less than the manufacturability score, thus the peptide passes the simulation. This process is repeated for each peptide in the set, eventually yielding a group of peptides predicted to pass the manufacturing simulation in each round. For illustration purposes, the immunogenicity sum presented in FIG. 4 is calculated only for the peptides shown. In practice, other peptides can also survive the simulation process, and the immunogenicity sum (or the probability of having at least one highly immunogenic peptide, calculated 1−Π(1−immunogenicityᵢ)) would include the immunogenicity scores for all peptides that pass the manufacturing simulation. These groups of peptides predicted to pass the manufacturing simulation can go through further refinement and processing as illustrated in FIG. 3.

Continuing with the discussion of FIG. 3, the peptides that pass each manufacturing simulation 320 are sent to the immunogenicity computing module 330, which calculates an immunogenicity score for the selection of peptides. In one embodiment, the number of peptides that successfully pass the manufacturing simulation module 320 can exceed the desired quantity intended for drug production. To manage this, the immunogenicity computing module 330 can select a subset from these peptides by ranking and selecting those with the highest immunogenicity scores. In one embodiment, selecting the subset from these peptides can involve consideration of a desired number of a type of peptide or desired mutations to be covered. In such scenarios, peptides that pass the manufacturing simulation can be first categorized by type or by target mutations. From each category, a number of top-performing peptides (e.g., peptides with greater immunogenicity scores or (immunogenicity×manufacturability) scores) can be selected to form the refined subset. In one embodiment, the selection of S peptides is based on both metric scores (such as immunogenicity scores or (immunogenicity×manufacturability) scores) and target mutations. For example, a peptide can be associated with a higher metric score such as an immunogenicity score. However, choosing this peptide can leave one mutation uncovered. In contrast, another peptide with a lower immunogenicity score can be capable of covering the mutation. Despite its lower immunogenicity score, the latter peptide might be the preferred choice because of the need to ensure that all mutations are effectively targeted, even if it means selecting a peptide with a lower metric score. This refined subset, which may be referred to herein as a subset of S peptides, is then used to calculate an aggregated immunogenicity score. In some embodiments, the number of peptides passing the manufacturing simulation can fall short of the desired quantity (such as less than S count). In such case, experts in the field can design and use heuristic methods to add additional peptides to reach the desired number of S peptides. With the refined subset of S peptides determined, the immunogenicity computing module 330 may calculate a total immunogenicity for the subset of S peptides. This iterative process, as shown in FIG. 3, can be performed for a number of times until a distribution of the aggregated immune score stabilizes. That is, the iteration continues until a median or mean of the aggregated immune score converges. In some embodiments, an ending criteria can be that a number of iterations is reached. Based on simulation results from a number of iterations, an expected immunogenicity can be computed for the collection of P peptides in the input 310.

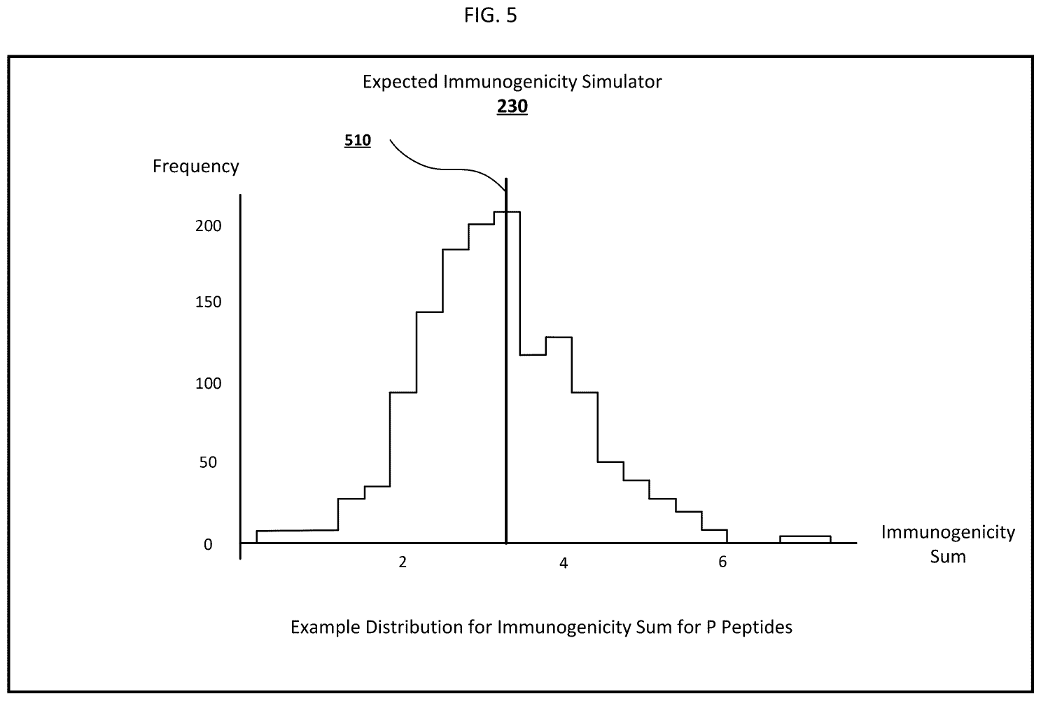

To illustrate with an example, consider the set of 48 peptides represented in input 310. These peptides can undergo a simulated manufacturing process conducted by the manufacturing simulation module 320. Following one iteration of simulation, 15 long peptides and 10 short peptides are predicted to be successfully manufactured. If a goal is to select 20 peptides for drug production, comprising a combination of 14 long and 6 short peptides, the immunogenicity computing module 330 can select the top 14 long peptides with the highest immune scores from the 15 available, and similarly select the top 6 short peptides with the highest immune scores from the 10 available. This selection forms the refined set of S peptides, including 14 long and 6 short peptides. The immunogenicity module 330 can then calculate an aggregated immunogenicity score by adding the immunogenicity score for the selected S peptides. This cycle starts again from input 310, and in each iteration, a new aggregated immunogenicity score is generated. After a series of such iterations, an expected immunogenicity score is determined for the group of 48 peptides. FIG. 5 displays an example of a distribution for aggregated immunogenicity scores generated from this simulation process.

FIG. 5 illustrates an exemplary histogram that results from conducting, for example, 1,000 iterations. The histogram is structured with the x-axis representing the sum of immunogenicity and the y-axis indicating the frequency of iterations. Each iteration contributes a data point to the histogram by computing an aggregated immunogenicity score for the S peptides. Upon completing 1000 iterations, the histogram in FIG. 5 visually represents the distribution of the immunogenicity sums. Based on this distribution, an expected immunogenicity sum is marked in the graph by a vertical line 510. In this example, the expected sum might be approximately 3.2. This expected immunogenicity sum is then considered the expected immunogenicity sum for the P peptides, or the 48 peptides in FIG. 4 as an example.

FIG. 6 illustrates an exemplary process for selecting a set of peptides from a larger pool of peptides utilizing a vaccine manufacturing simulator. The process begins with a substantial number of candidate peptides as input 610, potentially exceeding 100,000, which may be referred to as a pool of N peptides. The peptides in this pool are then evaluated and ordered by a peptide ranking module 620. A ranking module may rank the N peptides based on various criteria, such as immunogenicity or a compound metric such as the product of immunogenicity and manufacturability. Following the ranking process, the peptide ranking module 620 may select a preliminary set of P peptides for further refinement. The selection may also factor in one or more other constraints such as a minimum number of short/long peptides and one or more mutations to cover. In one embodiment, the peptide ranking module 620 may employ an optimization algorithm, such as a greedy substitution search, to identify the optimal set of P peptides. Each method of ranking, whether it is based on specific metrics or through optimization, is considered a ranking algorithm such as ranking algorithm A 630 and ranking algorithm B 631. For example, ranking algorithm A 630 may rank peptides based on the product of immunogenicity and manufacturability and other constraints such as short/long peptides and mutations to cover, while ranking algorithm B 631 may use a greedy search approach for selecting the P peptides. Such a vaccine manufacturing simulator is designed to leverage various algorithms, which allows for comparison of results and evaluation of effectiveness of each algorithm. The objective is to identify the algorithm that delivers the best performance in the context of vaccine manufacturing (e.g., maximize immunogenicity of the S peptides that are selected to become a manufactured drug product) while ensuring each and every mutation is covered by at least one peptide.

After the ranking and initial selection of P peptides, each of these peptides undergoes a series of iterative processes such as iterative process 690 and iterative process 691. These processes aim to calculate an expected sum of immunogenicity for the entire group of P peptides. Each iteration involves a manufacturing simulator, such as 640 and 641, which simulates the manufacturing process for the P peptides. For each round of simulation, a portion of P peptides survive the manufacturing simulation process and these passing peptides are predicted to be manufactured. If the number of peptides that pass exceeds the number needed for drug production, they can be reduced based on one or more constraints, such as choosing those with the highest immunogenicity ranking while ensuring a threshold number of short/long peptides that can cover each mutation and/or ensuring a threshold immunogenicity score for each mutation. The resulting refined subset of peptides may be referred to as a subset of S peptides. For this subset, a total immunogenicity score is calculated, which marks the completion of one iteration in the iterative processes 690 and 691.

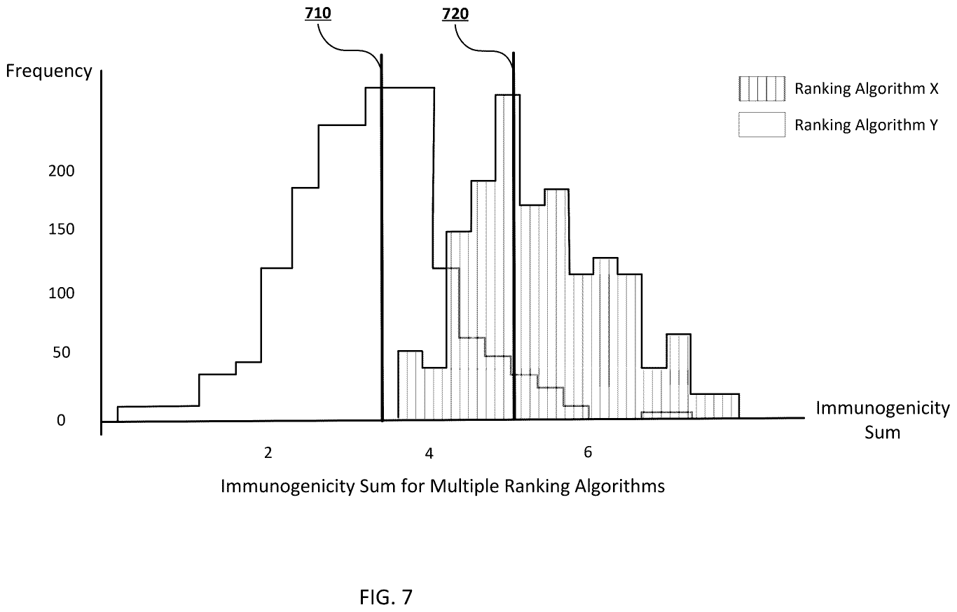

These iterations are repeated multiple times, potentially tens of thousands times or more, until a point of convergence is reached. Convergence can occur when the distribution stabilizes, such as when a mean or median of the distribution converges. When the iteration ends, an expected immunogenicity sum can be determined for the selected P peptides. Outcome from each iteration contributes to the overall results of a particular ranking algorithm, and multiple iterations of the ranking algorithm can be performed. For example, in the case of a greedy search ranking algorithm, each iteration might identify a different optimal set of P peptides. The vaccine manufacturing simulator continuously executes the ranking algorithm such as 630 and 631 and simulation process and generates an expected immunogenicity sum for each subset of refined S peptides. Using a ranking algorithm determination module 670, the ranking algorithm that exhibits the best performance can be chosen for selecting peptides in drug production. To illustrate the outcomes of applying different ranking algorithms in the vaccine manufacturing simulation, FIG. 7 depicts varied results from different distributions generated based on different ranking algorithms.

FIG. 7 illustrates an exemplary histogram with simulation results generated based on various ranking algorithms. The histogram illustrated in FIG. 7 is structured with the x-axis representing the sum of immunogenicity and the y-axis showing how often a particular immunogenicity sum occurred during the simulations. In such a histogram, there are two distributions represented by different outlines. One outlined with blank filling corresponds to the results from ranking algorithm Y, and the other with vertical lines indicates the results from ranking algorithm X. The histograms comprise bars of varying heights across the range of immunogenicity sums, where height associated with each bar reflects the frequency at which a particular sum was observed in the simulations. The results demonstrate a difference between the two ranking algorithms, where algorithm X tends to predict a group of peptides with a higher expected immunogenicity sum, around the value of 5, as seen by the vertical line 720. In contrast, the blank-filled pattern for algorithm Y indicates a lower expected immunogenicity sum, approximately 3.4, as indicated by the vertical line 710. From the results, it can be inferred that ranking algorithm X is more likely to select peptide groups with a greater cumulative immunogenicity compared to ranking algorithm Y. This may suggest that algorithm X is more effective for this specific goal, assuming a higher immunogenicity sum is desirable for efficacy of the vaccine production.

In one embodiment, a pooling algorithm is further implemented to the refined subset of S peptides for further improvement in vaccine efficacy. For example, the S peptides can be categorized into distinct pools, each intended to cover specific mutations or mutations. Within each pool, peptides are chosen not just for their high individual scores but also for how well they combine and interact with each other, such as synergistic or antagonistic effects. For example, peptides that enhance each other's effectiveness or stability when mixed are grouped together, while those that may hinder each other's performance are separated. As an example, certain peptides can be soluble on their own, but when mixed together, they can become insoluble. In such situations, these peptides might be allocated to separate pools because when they are combined together, they cannot be used in a drug product. The simulation can increase the probability that the peptides available for pooling will support multiple pooling configurations, so that (for instance) no highly immunogenic peptide need be excluded because there is no pool in which it can be placed.

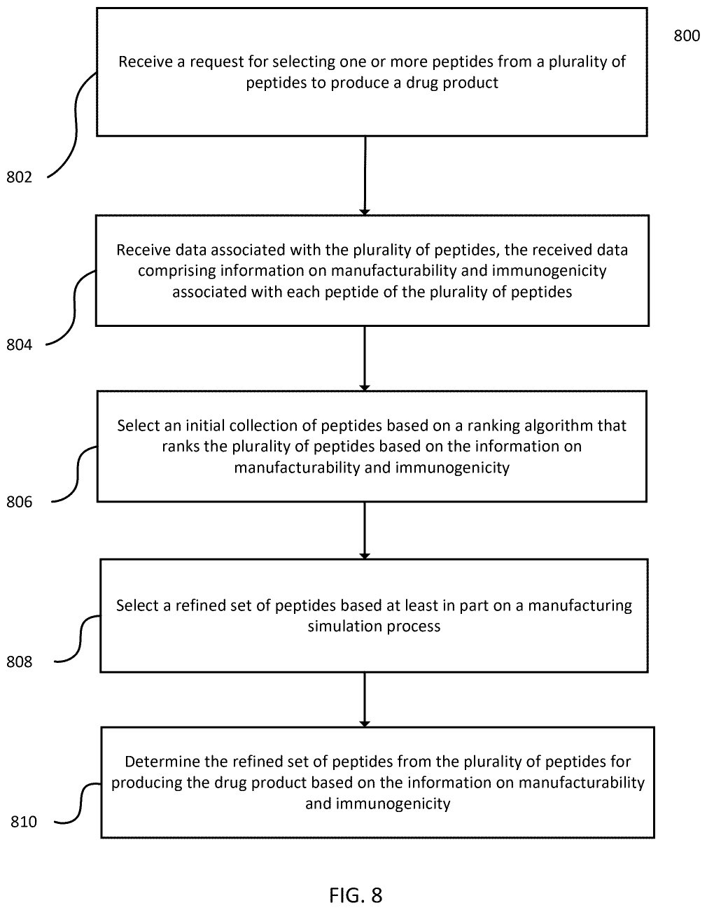

FIG. 8 illustrates an exemplary method 800 that can be used in accordance with the various embodiments described herein. It should be understood that for any process described herein there can be additional, fewer, or alternative steps performed in similar or alternative orders, or in parallel, within the scope of the various embodiments unless otherwise specifically stated.

The process 800 can initiate with receiving 802 a request to select one or multiple peptides from a pool of peptides for the creation of a drug product. Following this, a vaccine manufacturing simulation system can receive 804 or retrieve data associated with the peptides. This data includes information on manufacturability and immunogenicity associated with each peptide. The system can select 806 an initial collection of peptides based on a ranking algorithm that ranks the plurality of peptides based on the information on manufacturability and immunogenicity. To further refine the initial collection of peptides, the system can select 808 a refined set of peptides based at least in part on a manufacturing simulation process. The system can determine 810 the refined set of peptides from the plurality of peptides for producing the drug product based on the information on manufacturability and immunogenicity.

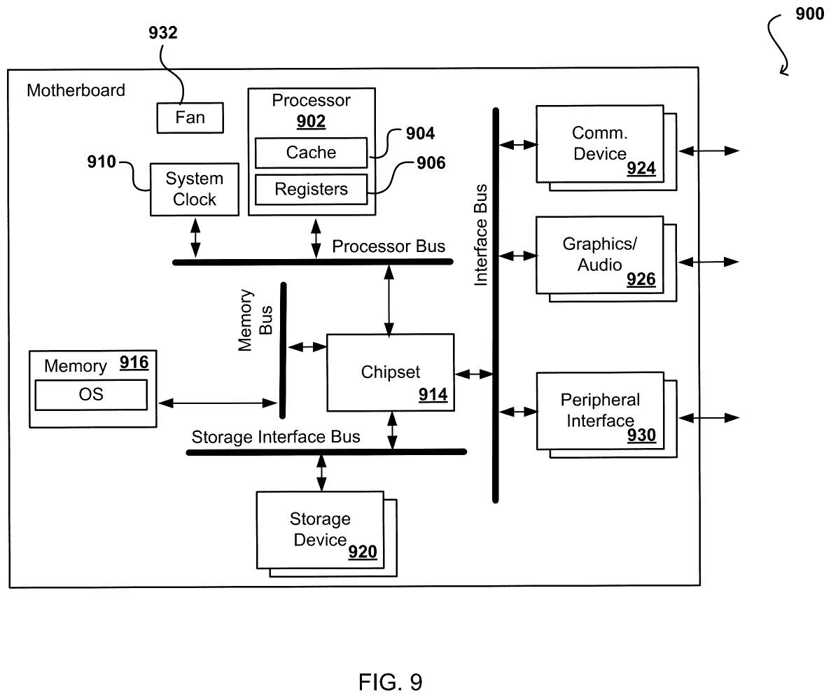

Computing resources, such as servers, that can have software and/or firmware updated in such a matter will generally include at least a set of standard components configured for general purpose operation, although various proprietary components and configurations can be used as well within the scope of the various embodiments. FIG. 9 illustrates components of an example computing device 900 that can be utilized in accordance with various embodiments. As known for computing devices, the computer will have one or more processors 902, such as central processing units (CPUs), graphics processing units (GPUs), and the like, that are electronically and/or communicatively coupled with various components using various buses, traces, and other such mechanisms. A processor 902 can include memory registers 906 and cache memory 904 for holding instructions, data, and the like. In this example, a chipset 914, which can include a northbridge and southbridge in some embodiments, can work with the various system buses to connect the processor 902 to components such as system memory 916, in the form or physical RAM or ROM, which can include the code for the operating system as well as various other instructions and data utilized for operation of the computing device. The computing device can also contain, or communicate with, one or more storage devices 920, such as hard drives, flash drives, optical storage, and the like, for persisting data and instructions similar, or in addition to, those stored in the processor and memory. The processor 902 can also communicate with various other components via the chipset 914 and an interface bus (or graphics bus, etc.), where those components can include communications devices 924 such as cellular modems or network cards, media components 926, such as graphics cards and audio components, and peripheral interfaces 930 for connecting peripheral devices, such as printers, keyboards, and the like. At least one cooling fan 932 or other such temperature regulating or reduction component can also be included as well, which can be driven by the processor or triggered by various other sensors or components on, or remote from, the device. Various other or alternative components and configurations can be utilized as well as known in the art for computing devices.

At least one processor 902 can obtain data from physical memory 916, such as a dynamic random access memory (DRAM) module, via a coherency fabric in some embodiments. It should be understood that various architectures can be utilized for such a computing device, that may include varying selections, numbers, and arguments of buses and bridges within the scope of the various embodiments. The data in memory may be managed and accessed by a memory controller, such as a DDR controller, through the coherency fabric. The data may be temporarily stored in a processor cache 904 in at least some embodiments. The computing device 900 can also support multiple I/O devices using a set of I/O controllers connected via an I/O bus. There may be I/O controllers to support respective types of I/O devices, such as a universal serial bus (USB) device, data storage (e.g., flash or disk storage), a network card, a peripheral component interconnect express (PCIe) card or interface 930, a communication device 924, a graphics or audio card 926, and a direct memory access (DMA) card, among other such options. In some embodiments, components such as the processor, controllers, and caches can be configured on a single card, board, or chip (i.e., a system-on-chip implementation), while in other embodiments at least some of the components may be located in different locations, etc.

An operating system (OS) running on the processor 902 can help to manage the various devices that may be utilized to provide input to be processed. This can include, for example, utilizing relevant device drivers to enable interaction with various I/O devices, where those devices may relate to data storage, device communications, user interfaces, and the like. The various I/O devices will typically connect via various device ports and communicate with the processor and other device components over one or more buses. There can be specific types of buses that provide for communications according to specific protocols, as may include peripheral component interconnect) PCI or small computer system interface (SCSI) communications, among other such options. Communications can occur using registers associated with the respective ports, including registers such as data-in and data-out registers. Communications can also occur using memory-mapped I/O, where a portion of the address space of a processor is mapped to a specific device, and data is written directly to, and from, that portion of the address space.

Such a device may be used, for example, as a server in a server farm or data warehouse. Server computers often have a need to perform tasks outside the environment of the CPU and main memory (i.e., RAM). For example, the server may need to communicate with external entities (e.g., other servers) or process data using an external processor (e.g., a General Purpose Graphical Processing Unit (GPGPU)). In such cases, the CPU may interface with one or more I/O devices. In some cases, these I/O devices may be special-purpose hardware designed to perform a specific role. For example, an Ethernet network interface controller (NIC) may be implemented as an application specific integrated circuit (ASIC) comprising digital logic operable to send and receive packets.

In an illustrative embodiment, a host computing device is associated with various hardware components, software components and respective configurations that facilitate the execution of I/O requests. One such component is an I/O adapter that inputs and/or outputs data along a communication channel. In one aspect, the I/O adapter device can communicate as a standard bridge component for facilitating access between various physical and emulated components and a communication channel. In another aspect, the I/O adapter device can include embedded microprocessors to allow the I/O adapter device to execute computer executable instructions related to the implementation of management functions or the management of one or more such management functions, or to execute other computer executable instructions related to the implementation of the I/O adapter device. In some embodiments, the I/O adapter device may be implemented using multiple discrete hardware elements, such as multiple cards or other devices. A management controller can be configured in such a way to be electrically isolated from any other component in the host device other than the I/O adapter device. In some embodiments, the I/O adapter device is attached externally to the host device. In some embodiments, the I/O adapter device is internally integrated into the host device. Also in communication with the I/O adapter device may be an external communication port component for establishing communication channels between the host device and one or more network-based services or other network-attached or direct-attached computing devices. Illustratively, the external communication port component can correspond to a network switch, sometimes known as a Top of Rack (“TOR”) switch. The I/O adapter device can utilize the external communication port component to maintain communication channels between one or more services and the host device, such as health check services, financial services, and the like.

The I/O adapter device can also be in communication with a Basic Input/Output System (BIOS) component. The BIOS component can include non-transitory executable code, often referred to as firmware, which can be executed by one or more processors and used to cause components of the host device to initialize and identify system devices such as the video display card, keyboard and mouse, hard disk drive, optical disc drive and other hardware. The BIOS component can also include or locate boot loader software that will be utilized to boot the host device. For example, in one embodiment, the BIOS component can include executable code that, when executed by a processor, causes the host device to attempt to locate Preboot Execution Environment (PXE) boot software. Additionally, the BIOS component can include or takes the benefit of a hardware latch that is electrically controlled by the I/O adapter device. The hardware latch can restrict access to one or more aspects of the BIOS component, such controlling modifications or configurations of the executable code maintained in the BIOS component. The BIOS component can be connected to (or in communication with) a number of additional computing device resources components, such as processors, memory, and the like. In one embodiment, such computing device resource components may be physical computing device resources in communication with other components via the communication channel. The communication channel can correspond to one or more communication buses, such as a shared bus (e.g., a processor bus, a memory bus), a point-to-point bus such as a PCI or PCI Express bus, etc., in which the components of the bare metal host device communicate. Other types of communication channels, communication media, communication buses or communication protocols (e.g., the Ethernet communication protocol) may also be utilized. Additionally, in other embodiments, one or more of the computing device resource components may be virtualized hardware components emulated by the host device. In such embodiments, the I/O adapter device can implement a management process in which a host device is configured with physical or emulated hardware components based on a variety of criteria. The computing device resource components may be in communication with the I/O adapter device via the communication channel. In addition, a communication channel may connect a PCI Express device to a CPU via a northbridge or host bridge, among other such options.

In communication with the I/O adapter device via the communication channel may be one or more controller components for managing hard drives or other forms of memory. An example of a controller component can be a SATA hard drive controller. Similar to the BIOS component, the controller components can include or take the benefit of a hardware latch that is electrically controlled by the I/O adapter device. The hardware latch can restrict access to one or more aspects of the controller component. Illustratively, the hardware latches may be controlled together or independently. For example, the I/O adapter device may selectively close a hardware latch for one or more components based on a trust level associated with a particular user. In another example, the I/O adapter device may selectively close a hardware latch for one or more components based on a trust level associated with an author or distributor of the executable code to be executed by the I/O adapter device. In a further example, the I/O adapter device may selectively close a hardware latch for one or more components based on a trust level associated with the component itself. The host device can also include additional components that are in communication with one or more of the illustrative components associated with the host device. Such components can include devices, such as one or more controllers in combination with one or more peripheral devices, such as hard disks or other storage devices. Additionally, the additional components of the host device can include another set of peripheral devices, such as Graphics Processing Units (“GPUs”). The peripheral devices and can also be associated with hardware latches for restricting access to one or more aspects of the component. As mentioned above, in one embodiment, the hardware latches may be controlled together or independently.

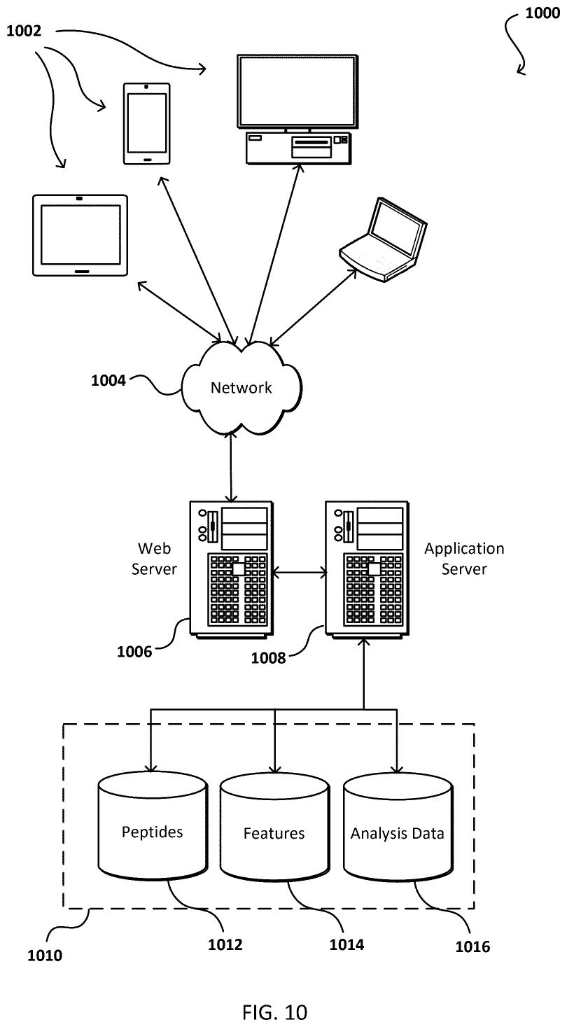

As discussed, different approaches can be implemented in various environments in accordance with the described embodiments. For example, FIG. 10 illustrates an example of an environment 1000 for implementing aspects in accordance with various embodiments. As will be appreciated, although a Web-based environment is used for purposes of explanation, different environments may be used, as appropriate, to implement various embodiments. The system includes an electronic client device 1002, which can include any appropriate device operable to send and receive requests, messages or information over an appropriate network 1004 and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server 1006 for receiving requests and serving content in response thereto, although for other networks, an alternative device serving a similar purpose could be used, as would be apparent to one of ordinary skill in the art.

The illustrative environment includes at least one application server 1008 and a data store 1010. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 1008 can include any appropriate hardware and software for integrating with the data store 1010 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the Web server 1006 in the form of HTML, XML or another appropriate structured language in this example. The handling of all requests and responses, as well as the delivery of content between the client device 1002 and the application server 1008, can be handled by the Web server 1006. It should be understood that the Web and application servers are not required and are merely example components, as structured code discussed herein can be executed on any appropriate device or host machine as discussed elsewhere herein.

The data store 1010 can include several separate data tables, databases or other data storage mechanisms and media for storing data relating to a particular aspect. For example, the data store illustrated includes mechanisms for storing peptides 1012 or peptide representations and analysis data 1016, which can be used to serve content for the production side. The data store is also shown to include a mechanism for storing feature data for the peptides 1014. It should be understood that there can be many other aspects that may need to be stored in the data store, such as manufacturing data or other training data, which can be stored in any of the above listed mechanisms as appropriate or in additional mechanisms in the data store 1010. The data store 1010 is operable, through logic associated therewith, to receive instructions from the application server 1008 and obtain, update or otherwise process data in response thereto. In one example, a user might submit a request for peptide synthesis. In this case, the data store might be used to access the peptide information, feature data, and analysis data to obtain information as to whether the peptide is synthesizable. The information can then be returned to the user, such as in a results listing on a Web page that the user is able to view via a browser on the user device 1002.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 10. Thus, the depiction of the system 1000 in FIG. 10 should be taken as being illustrative in nature and not limiting to the scope of the disclosure.

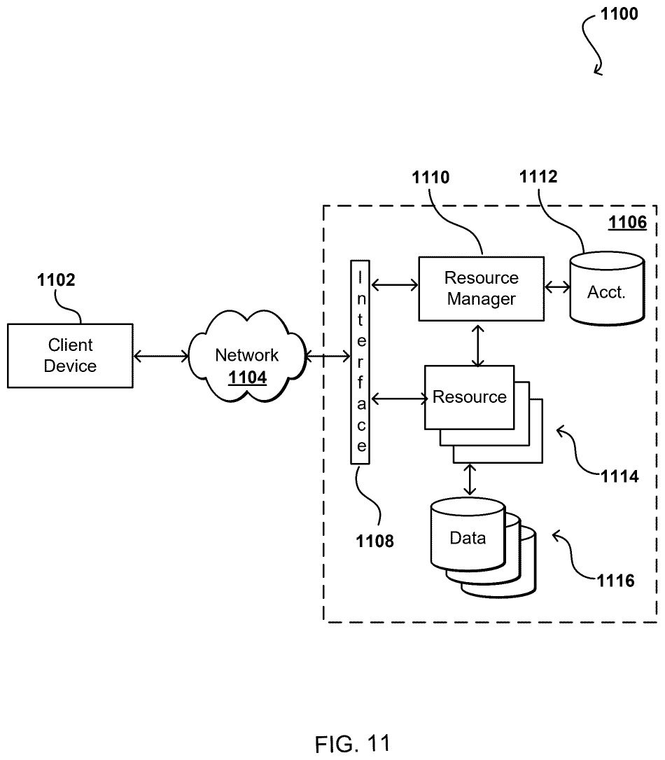

FIG. 11 illustrates an example environment 1100 in which aspects of the various embodiments can be implemented. In this example a user is able to utilize a client device 1102 to submit requests across at least one network 1104 to a multi-tenant resource provider environment 1106. The client device can include any appropriate electronic device operable to send and receive requests, messages, or other such information over an appropriate network and convey information back to a user of the device. Examples of such client devices include personal computers, tablet computers, smart phones, notebook computers, and the like. The at least one network 1104 can include any appropriate network, including an intranet, the Internet, a cellular network, a local area network (LAN), or any other such network or combination, and communication over the network can be enabled via wired and/or wireless connections. The resource provider environment 1106 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 1114 of one or more types. These types can include, for example, application servers operable to process instructions provided by a user or database servers operable to process data stored in one or more data stores 1116 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 1114 can submit a request that is received to an interface layer 1108 of the provider environment 1106. The interface layer can include application programming interfaces (APIs) or other exposed interfaces enabling a user to submit requests to the provider environment. The interface layer 1108 in this example can also include other components as well, such as at least one Web server, routing components, load balancers, and the like. When a request to provision a resource is received to the interface layer 1108, information for the request can be directed to a resource manager 1110 or other such system, service, or component configured to manage user accounts and information, resource provisioning and usage, and other such aspects. A resource manager 1110 receiving the request can perform tasks such as to authenticate an identity of the user submitting the request, as well as to determine whether that user has an existing account with the resource provider, where the account data may be stored in at least one data store 1112 in the provider environment. A user can provide any of various types of credentials in order to authenticate an identity of the user to the provider. These credentials can include, for example, a username and password pair, biometric data, a digital signature, or other such information. The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options.

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 1102 to communicate with an allocated resource without having to communicate with the resource manager 1110, at least until such time as a relevant aspect of the user account changes, the user is no longer granted access to the resource, or another such aspect changes.

The resource manager 1110 (or another such system or service) in this example can also function as a virtual layer of hardware and software components that handles control functions in addition to management actions, as may include provisioning, scaling, replication, etc. The resource manager can utilize dedicated APIs in the interface layer 1108, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 1108 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
receiving a request for selecting one or more peptides from a plurality of peptides to produce a drug product;
receiving data comprising information on manufacturability associated with each peptide of the plurality of peptides;
receiving data comprising information on immunogenicity associated with each peptide of the plurality of peptides; and
generating, using a statistical model pipeline, a set of peptides from the plurality of peptides for producing the drug product based on the information on manufacturability and the information on a second feature, wherein the statistical model optimizes at least one of an immunogenicity sum or a manufacturability sum associated with the set of peptides while meeting one or more criteria.

2. The computer-implemented method of claim 1, wherein generating the set of peptides further comprises:
ranking the plurality of peptides based on the information on manufacturability and the information on the second feature associated with each peptide, wherein the second feature is immunogenicity associated with each peptide; and
selecting, based on the ranking and the one or more criteria, a first set of peptides from the plurality of peptides for inclusion in a simulated manufacturing process, the simulated manufacturing process being a part of the statistical model pipeline, wherein the one or more criteria include one or more of an expected number of long peptides, an expected number of short peptides, and one or more specific target mutations that the drug is intended to be effective against.

3. The computer-implemented method of claim 2, further comprising:
generating, based on the simulated manufacturing process, a second set of peptides, wherein said second set of peptides is predicted to pass the simulated manufacturing process;
selecting a subset of peptides from the second set of peptides based on an immunogenicity score associated with each peptide and based on the one or more criteria; and
calculating an aggregated immunogenicity score for the subset of peptides.

4. The computer-implemented method of claim 3, further comprising:
repeatedly executing the simulated manufacturing process on the first set of peptides for a number of iterations, producing the number of simulation outcomes; and
determining an average aggregated immunogenicity score associated with the first set of peptides based on the number of simulation outcomes.

5. The computer-implemented method of claim 2, wherein ranking the plurality of peptides further comprises:
computing a product of immunogenicity score and manufacturability score for each peptide, wherein the information on immunogenicity includes an immunogenicity score for each peptide and the information on manufacturability includes a manufacturability score for each peptide; and
wherein the ranking is based on the product of immunogenicity score and manufacturability score.

6. A computer-implemented method, comprising:
receiving a request for selecting one or more peptides from a plurality of peptides to produce a drug product;
receiving data associated with the plurality of peptides, the received data comprising information on manufacturability and information on one or more features associated with each peptide of the plurality of peptides; and
determining, using a statistical model, a set of peptides from the plurality of peptides for producing the drug product based on the information on manufacturability and the information on at least one of the one or more features.

7. The computer-implemented method of claim 6, wherein the information on one or more features include one or more of: information on immunogenicity, information on expected numbers of different types of peptides, and information on specific target mutations that the drug is intended to be effective against associated with each peptide.

8. The computer-implemented method of claim 6, wherein determining the set of peptides further comprises:
ranking the plurality of peptides based on the information on manufacturability and the information on the feature associated with each peptide; and
selecting, based on the ranking and one or more criteria, a first set of peptides from the plurality of peptides for inclusion in a simulated manufacturing process.

9. The computer-implemented method of claim 8, wherein the criteria includes one or more of an expected number of long peptides, an expected number of short peptides, and one or more specific target mutations that the drug is intended to be effective against.

10. The computer-implemented method of claim 8, further comprising:
generating, based on the simulated manufacturing process, a second set of peptides, wherein said second set of peptides is predicted to pass the simulated manufacturing process;
selecting a subset of peptides from the second set of peptides based on a feature score associated with each peptide and based on the one or more criteria; and
calculating an aggregated feature score for the subset of peptides.

11. The computer-implemented method of claim 10, further comprising:
repeatedly executing the simulated manufacturing process on the first set of peptides for a number of iterations, producing the number of simulation outcomes; and
determining an average aggregated feature score associated with the first set of peptides based on the number of simulation outcomes.

12. The computer-implemented method of claim 8, wherein ranking the plurality of peptides further comprises:
computing a product of feature score and manufacturability score for each peptide, wherein the information on at least one of the features includes a feature score for each peptide and the information on manufacturability includes a manufacturability score for each peptide; and
wherein the ranking is based on the product of feature score and manufacturability score.

13. The computer-implemented method of claim 8, wherein selecting the set of peptides is based on a greedy search algorithm that optimizes a goal associated with the set of peptides while meeting one or more criteria.

14. A computing system, comprising:
a computing device processor; and
a memory device including instructions that, when executed by the computing device processor, enable the computing system to:
receive a request for selecting one or more peptides from a plurality of peptides to produce a drug product;
receive data associated with the plurality of peptides, the received data comprising information on manufacturability and one or more features associated with each peptide of the plurality of peptides; and
determine, using a statistical model, a set of peptides from the plurality of peptides for producing the drug product based on the information on manufacturability and at least one of the features.

15. The computing system of claim 14, wherein the one or more features includes one or more of: information on immunogenicity, information on expected numbers of different types of peptides, and information on specific target mutations that the drug is intended to be effective against.

16. The computing system of claim 14, wherein the instructions further enable the computing system to:
rank the plurality of peptides based on the information on manufacturability and another feature associated with each peptide; and
select, based on the ranking, a first set of peptides from the plurality of peptides for inclusion in a simulated manufacturing process.

17. The computing system of claim 14, wherein the instructions further enable the computing system to:
generate, based on the simulated manufacturing process, a second set of peptides predicted to pass the simulated manufacturing process;
select a subset of peptides from the second set based on a feature score associated with each peptide; and
calculate an aggregated feature score for the subset of peptides.

18. The computing system of claim 14, wherein the instructions further enable the computing system to:
repeatedly execute the simulated manufacturing process on the first set of peptides for a number of iterations, producing the number of simulation outcomes; and
generate an average aggregated feature score associated with the first set of peptides based on the number of simulation outcomes.

19. The computing system of claim 15, wherein ranking the plurality of peptides further comprises instructions that enable the computing system to:
compute a product of a feature score and a manufacturability score for each peptide, wherein the feature score and manufacturability score are in the received data for each peptide; and
wherein the ranking is based on the product of feature score and manufacturability score.

20. The computing system of claim 15, wherein generating the set of peptides includes selecting the peptides for one or more criteria specified in the received request, wherein the one or more criteria are selected from the group consisting of: an expected number of long peptides, an expected number of short peptides, and one or more specific target mutations that the drug is intended to be effective against.
